(19) United States
(12) Patent Application Publication
(10) Pub. No.: US 2026/0265344 A1
(43) Pub. Date: Sep. 10, 2026

(54) BREVIBACILLUS LATEROSPORUS AND METHOD FOR EXTRACTING COLLAGEN/COLLAGEN PEPTIDES USING BREVIBACILLUS LATEROSPORUS

(71) Applicant: Barel(Guangzhou)New Material Technology Co.,Ltd, Guangzhou (CN)

(72) Inventors: Xiaomei Zhang, Guangzhou (CN); Hao Liu, Guangzhou (CN); Xue Ou, Shanghai (CN); Yisen Dong, Guangzhou (CN)

(21) Appl. No.: 19/554,882

(22) Filed: Mar. 3, 2026

(30) Foreign Application Priority Data
Mar. 4, 2025 (CN) 202510244930.X

(51) Int. Cl. C07K 14/78; A61K 8/65; C12N 1/205; C12N 9/64; C12P 21/02; C12P 21/06; C12R 1/01

(52) U.S. Cl. CPC C07K 14/78; A61K 8/65; C12N 1/205; C12N 9/6491; C12P 21/02; C12P 21/06; C12R 2001/01; C12Y 304/24003

(57) ABSTRACT

Provided is a Brevibacillus laterosporus SCUT-BR-2 capable of producing collagen protease and a use in an extraction of animal collagen/collagen peptides. Specifically, it includes: screening strains that produce collagen protease from meat products, conducting morphological observation, physiological and biochemical experiments, and measuring, and using collagen enzyme crude extract of the strain to extract collagen from animal skin, and detecting a relative molecular weight of collagen; at the same time, the crude extract was used to decompose bovine bone collagen protein, obtaining a bovine bone collagen peptide with good antioxidant properties, anti-wrinkle, firming, and soothing moisturizing effects.

### CROSS-REFERENCE TO RELATED APPLICATIONS

This application claims priority to Chinese Patent Application No. 202510244930.X, filed on Mar. 4, 2025, which is hereby incorporated by reference in its entirety.

### SEQUENCE LISTING

The present application contains a sequence listing which was filed electronically in XML format and is hereby incorporated by reference in its entirety. Besides, the XML copy is created on Apr. 27, 2026, is named “PA-2026011562-US-Sequence Listing” and is 3,409 bytes in sizes.

### TECHNICAL FIELD

The present disclosure relates to the field of microbial fermentation cultivation and utilization of microorganisms for collagen hydrolysis technologies, and in particular, to a Brevibacillus laterosporus and a method for extracting collagen/collagen peptides using Brevibacillus laterosporus.

### BACKGROUND

Brevibacillus laterosporus is a type of facultative anaerobic bacterium with rod-shaped, peri flagellar cells, and is commonly used as a probiotic for biological control. The use of Brevibacillus laterosporus is very extensive. In animal husbandry, it can be used as a microbial additive in animal feed. In agriculture, as a microbial fertilizer, it can effectively improve soil fertility and promote crop growth. In the fishing industry, Brevibacillus laterosporus can inhibit the photosynthesis of algal cells, rendering it difficult for them to survive, and cause lipid peroxidation of algal cell membranes, leading to algal death and achieving water quality control and blue-green algae control, which is beneficial for aquaculture. In the field of food, the antimicrobial peptides produced by it have broad-spectrum antibacterial activity, high temperature resistance, acid and alkali resistance, and sensitivity to proteolytic enzymes, which can be applied to food preservation and freshness preservation.

Collagen is a three-helix fibrous protein composed of three peptide chains twisted together. It is an insoluble protein in fibrous proteins and is the most abundant protein among many vertebrates and invertebrates. Collagen has good low immunity, biocompatibility, and degradability, and can be widely used in fields such as food, cosmetics, and medicine. Collagen peptides (also known as collagen peptides) are relatively small-molecular-weight products formed by the hydrolysis of collagen. Compared to collagen, collagen peptides have higher absorption and utilization rates, better effects, and can promote the absorption of other proteins in food. Collagen peptides can be completely dissolved in water (or cold water), and also have certain anti-hypertensive and angiotensin (ACE) inhibitory activities, thereby improving skin and wound healing, antibacterial and antioxidant activities, promoting bone synthesis, etc. Therefore, collagen peptides play an irreplaceable and important role in industries such as food (including health products), medical care, beauty and skincare, and their demand is increasing day by day.

Currently, collagen is mainly extracted from animal skins using various extraction methods, including alkaline extraction, acid extraction, hot water extraction, enzymatic extraction, and composite methods. Extraction by acid alone or enzyme acid combination method can easily damage protein structure, while alkaline extraction often causes protein racemization. The method of extracting collagen by hot water extraction generally leads to incomplete decomposition of collagen tissue and lower collagen yield. Compared to other methods, enzymatic extraction is milder and more efficient, with better retention of product activity. In the existing technology, such as CN113943770B, the method of extracting fish skin collagen using acid plus enzyme combination plus salt precipitation is complex and uses commercial enzymes, resulting in high costs. CN110922475A discloses that two or more combined enzymes are used to extract collagen from pig skin, under conditions of pH 2-3 and temperature 30-40° C. during gastric protease treatment. When treated with papain, the pH is 6-7 and the temperature is 50-60° C. When treated with trypsin, the pH is 7-8 and the temperature is 40-50° C. In existing methods, commercial enzymes are used for enzymatic hydrolysis. However, commercial enzymes are costly, and the reaction conditions are based on the optimal enzyme activity. Therefore, the use of commercial enzymes makes the entire collagen extraction process costly and complicated.

There are currently two main methods for obtaining collagen peptides from further hydrolysis of collagen: synthesis and decomposition. Scholars have used synthetic methods to produce bovine bone collagen peptides, but this method requires first determining the sequence of bovine bone collagen peptides, which have a high threshold and the decomposition method is relatively easy. The commonly used enzymatic hydrolysis methods include pepsin, alkaline protease, neutral protease, trypsin, animal complex protease, flavor protease, and papain. Similarly, in the process of degrading molecular collagen peptides, the current mainstream method is also to directly use commercial enzymes. As mentioned earlier, these mature commercial enzymes are not only very expensive, but also have harsh degradation conditions (requiring optimal reaction conditions for enzyme activity to achieve the best degradation effect and avoid wasting enzymes, so there are many requirements for temperature, substrate concentration, etc.), and the amount used is large, which undoubtedly increases the production cost of enterprises.

Therefore, it is crucial to develop a bacterium that can naturally produce proteolytic enzymes to replace the expensive and large-scale commercial enzymes mentioned above. By combining a simple fermentation and self-produced enzyme process, this technology can be used to extract collagen and/or obtain collagen peptides, reduce extraction costs, simplify extraction processes, lower equipment requirements, and reduce environmental pollution. This is essential for enterprises to scale up the preparation and widespread application of collagen/collagen peptides, and is also a new method for achieving higher resource utilization of fermentation process products, thereby providing new references and ideas for environmental protection and product treatment of fermentation enterprises.

### SUMMARY

In order to solve the practical technical problems in the production process of many enterprises mentioned above, the present disclosure has developed a new type of Brevibacillus laterosporus, and thus obtained a method for directly extracting collagen/obtaining collagen peptides using a fermentation product of this Brevibacillus laterosporus. The specific technical solution is as follows.

A type of Brevibacillus laterosporus, named as Brevibacillus laterosporus SCUT-BR-2, was deposited on Oct. 15, 2024, it was deposited at the Guangdong institute of microbiology, with an address at 5th Floor, No. 59 Building, No. 100 Xianlie Zhong Road, Yuexiu District, Guangzhou 510070, PR China and with a deposit number of GDMCC No. 65271.

In some embodiments of the present disclosure, the Brevibacillus laterosporus strain is isolating, culturing, and purifying Brevibacillus laterosporus from meat products.

In some embodiments of the present disclosure, a culture medium used for Brevibacillus laterosporus includes a LB medium, a medium A, and a beef extract peptone medium.

In some embodiments of the present disclosure, a formula of the culture medium A is: collagen powder 0.8%, beef paste 0.1%, glucose 0.1%, KH₂PO₄ 0.05%, MgSO₄·7H₂O 0.01%, Agar 2%, adjusting a pH of the medium A to 7.0.

In some embodiments of the present disclosure, steps of isolating, culturing, and purifying Brevibacillus laterosporus from meat products include: grinding the meat products, weighing 1 g and adding it to a triangular flask containing 49 mL sterile physiological saline, shaking thoroughly for 30 minutes, standing still, taking a middle supernatant as a bacterial suspension, and performing gradient dilution, gradient diluted bacterial suspension was separately coated onto a LB agar solid medium using plate dilution separation method, and cultured in a constant temperature incubator at 30-40° C. until single colonies grew, selecting single colonies and placing on a screening medium A plate, and inverting and cultivating for 1-2 days at 30-40° C., based on screening characteristics, selecting single colonies with normal growth and significant differences in colony morphology and repeatedly streaking on beef extract peptone medium for pure culture, after numbering, they were stored for future use.

The present disclosure further applies for protection of a method for enzymatic hydrolysis of bovine bone collagen using the aforementioned Brevibacillus laterosporus SCUT-BR-2, including: preparing a 12.5% bovine bone collagen solution, stirring and dissolving at 50° C., adding 0.3% (based on a mass of bovine bone collagen) of collagen enzyme crude extract, reacting at 50° C. for 8-24 hours, then adding 0.5%-3% activated carbon powder (based on the mass of bovine bone collagen) to a reaction solution, adsorbing and deodorizing for 1 hour, filtering once with Double Circle brand qualitative rapid filter paper, and then filtering once with a 0.45 μm microporous filter membrane to obtain a clear and transparent filtrate; heating the filtrate at 70° C. for 5 min to inactivate an enzyme, collecting the filtrate for spray drying to obtain the bovine bone collagen peptide.

In some embodiments of the present disclosure, the obtained bovine bone collagen peptide satisfies at least one of the following properties:
(1) a scavenging rate of bovine bone collagen peptide on DPPH free radicals is 10.53±0.47%;
(2) an inhibition rate of elastase is 14.39±0.52%;
(3) an inhibition rate of hyaluronidase is 47.02±2.49%;
(4) a weight average molecular weight of bovine bone collagen peptide is 1870 g/mol.

In some embodiments of the present disclosure, the collagen enzyme crude extract is obtained by the following method: picking a certain amount of Brevibacillus laterosporus SCUT-BR-2 colonies from a beef extract peptone medium plate, adding sterile water with a bacterial mass of 1.5 times, shaking well, and centrifuging at 4° C. and 8000 rmp for 10 minutes, an obtained supernatant is the collagen enzyme crude extract used in the present disclosure.

In some embodiments of the present disclosure, a yield of the obtained bovine bone collagen peptide is 42.48%.

The present application further requests protection for a use of the aforementioned Brevibacillus laterosporus SCUT-BR-2 in an extraction of collagen from fish skin, pig skin, cowhide, and donkey skin.

### Beneficial Technical Effects

The Brevibacillus laterosporus SCUT-BR-2 provided by the present disclosure has a good effect in the collagen extraction process of animal skin such as fish skin. Meanwhile, when using bovine bone collagen as raw material, its ability to break down collagen into small molecule peptides is significant. Specifically, the obtained bovine bone collagen peptide showed a DPPH free radical scavenging rate of 10.53±0.47%, an elastase inhibition rate of 14.39±0.52%, and a hyaluronidase inhibition rate of 47.02±2.49%, indicating that bovine bone collagen peptide has certain antioxidant, anti-wrinkle and firming effects, and soothing and moisturizing effects, and can be well applied in various skincare products, beauty solutions, etc. The enzyme extracted from Brevibacillus laterosporus SCUT-BR-2 provided by the present disclosure is highly suitable for extracting and processing collagen under neutral conditions, avoiding the need for additional acid-base reagents to adjust acidity and alkalinity of commercial enzymes such as gastric protease or alkaline protease, which can easily introduce inorganic ion impurities. Besides that, the present disclosure directly uses the crude fermentation extract of B. laterosporus SCUT-BR-2, i.e., the collagen crude extract used in the present disclosure, for the extraction of collagen and the treatment of bovine bone collagen peptides. This not only omits steps such as enzyme purification, but also comprehensively utilizes other beneficial components in the crude extract that promote enzymatic hydrolysis besides the extracellular collagenase produced by B. laterosporus SCUT-BR-2 itself, such as the extracellular secretions produced by B. laterosporus SCUT-BR-2 during the fermentation process. The presence of these components can provide a beneficial solution for the crude extract. The collagen enzyme provides certain vitality and nutrients, which promote the activity of the enzyme and make its enzymatic hydrolysis more efficient. The promotion of extracellular enzyme activity by extracellular secretions is mainly reflected in enhancing enzyme stability. Certain extracellular secretions can provide enzymes with a relatively stable and familiar living and working environment, thereby enhancing their stability and activity. Secondly, the extracellular secretions in the crude extract itself provide familiar substrates for maintaining enzyme activity, promoting the reaction rate of the enzyme. The applicant has found that the B. laterosporus SCUT-BR-2 provided in the present disclosure can enhance the degradation efficiency of substrates by extracellular enzymes by secreting specific polysaccharides or proteins. Besides that, different components in the extracellular secretions of bacteria in the crude extract will interact with each other to enhance the catalytic effect of enzymes. In the present disclosure, small molecules secreted by Brevibacillus laterosporus SCUT-BR-2 can activate enzyme activity or enhance substrate accessibility, thereby accelerating the reaction rate of enzymatic hydrolysis. From the perspective of processing cost, the cultivation conditions of the SCUT-BR-2 provided by the present disclosure are simpler than those of commercially available enzymes, easier to preserve, and have low cost. It is also easy to industrialize and has good prospects for development and application. The crude extract of the enzyme omits a series of tedious steps such as purification and separation, simplifying the entire process flow and comprehensively utilizing the various components in the crude extract, greatly reducing production costs, saving resources, and reducing environmental pollution caused by wastewater during the production process and cost of treating wastewater. Meanwhile, the B. laterosporus SCUT-BR-2 and its crude extract provided by the present disclosure also have important application potential in solving environmental pollution and resource waste problems. By applying it to extract collagen from animal skin, it is possible to comprehensively utilize animal skin resources and obtain high-value products, thereby providing a sustainable solution for the application of biotechnology and promoting the green development of biotechnology.

### BRIEF DESCRIPTION OF DRAWINGS

FIG. 1 is a colony morphology of SCUT-BR-2.

FIG. 2 is a gram staining image of SCUT-BR-2.

FIG. 3 is a 16S rDNA sequence diagram of SCUT-BR-2.

FIG. 4 is a phylogenetic tree of SCUT-BR-2.

FIG. 5 is fish skin collagen protein after freeze-drying (unpolished).

FIG. 6 is cowhide collagen after freeze-drying (unpolished).

FIG. 7 is pig skin collagen after freeze-drying (unground).

FIG. 8 is donkey skin collagen after freeze-drying (unpolished).

FIG. 9 is infrared spectrum of collagen protein.

FIG. 10 is a Circular Dichroism (CD) spectrum of collagen.

FIG. 11 is a bovine bone collagen peptide after spray drying.

### DESCRIPTION OF EMBODIMENTS

Below, the technical solutions of each embodiment of the present disclosure will be clearly and completely described by combining the technical solutions of the present disclosure with different specific embodiments. Obviously, the described embodiments are only a part of the embodiments of the present disclosure, not all of them. Based on the embodiments of the present disclosure, all other embodiments obtained by those skilled in the art without creative work are within the protection scope of the present disclosure. The experimental methods described in the following examples, unless otherwise specified, are all conventional methods. Unless otherwise specified, the reagents and materials can be obtained through ordinary commercial channels.

The present disclosure obtains a novel strain of Brevibacillus laterosporus SCUT-BR-2 through cultivation in a culture medium and subsequent screening and isolation. The strain was deposited on Oct. 15, 2024 at the Guangdong Microbial Culture Collection Center, located on the 5th floor of Building 59, No. 100 Xianlie Middle Road, Guangzhou City, Guangdong Province, with a deposit number of GDMCC No. 65271. The Brevibacillus laterosporus described was isolated and purified from meat products. The meat product mentioned is a pork product.

In a screening step of Brevibacillus laterosporus SCUT-BR-2, a culture media used includes a LB medium, a medium A, and a beef extract peptone medium. A formula for the medium A is: collagen powder 0.8%, beef paste 0.1%, glucose 0.1%, KH₂PO₄ 0.05%, MgSO₄·7H₂O 0.01%, Agar 2%, adjusting a pH of the medium A to 7.0. The acquisition of this culture medium is based on 1 liter of sterile water or purified water, in which the various proportions of substances in the above formula are added and the pH is adjusted to 7.0.

The method for isolating, culturing, and purifying Brevibacillus laterosporus from meat products include:
grinding the pork product, weighing 1 g and adding it to a triangular flask containing 49 mL sterile physiological saline, shaking well for 30 minutes, letting it stand, and taking a middle supernatant as a bacterial suspension for gradient dilution, the gradient diluted bacterial suspension was separately coated onto a LB agar solid medium using plate dilution separation method, and cultured in a constant temperature incubator at 30-40° C. until single colonies grew. Single colonies were selected and placed on screening medium plates, and inverted at 30-40° C. for 1-2 days. Based on screening characteristics such as single colony size, surface structure, texture, luster, and color, single colonies with normal growth and significant differences in colony morphology were selected and repeatedly streaked on beef extract peptone medium for pure culture. After numbering, they were stored for future use.

The preferred temperature for the above-mentioned constant temperature incubator is 35° C., and the preferred temperature for the inverted culture is 35° C. The applicant was pleasantly surprised to find that the cultivation effect was better under optimal conditions.

### Bacterial Detection

### (1) Morphological Identification

Follow the experimental methods in the “Manual for Identification of Common Bacterial Systems” and record the colony morphology, staining characteristics, and optical microscope morphology.

### (2) Physiological and Biochemical Identification

According to the physiological and biochemical indicators in the “Manual for Identification of Common Bacterial Systems”, various strains were tested for physiological and biochemical indicators such as enzyme contact, V-P reaction, methyl red reaction, nitrate reduction test, etc.

(3) 16S rDNA Sequence Analysis

Sending separated bacteria to Sangon Biotech (Shanghai) Co., Ltd. for sequencing on an inclined surface of a test tube (beef extract peptone medium). Comparing the sequencing results of 16S rDNA of bacterial strains using NCBI database and constructing a phylogenetic tree.

Morphological Identification of Brevibacillus laterosporus

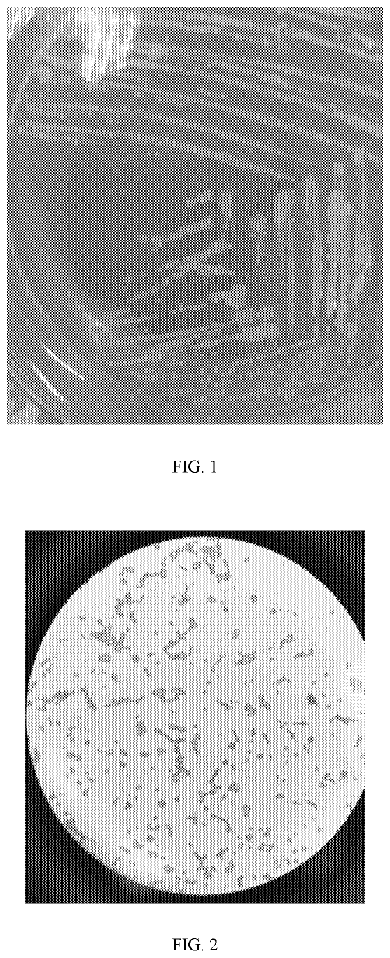

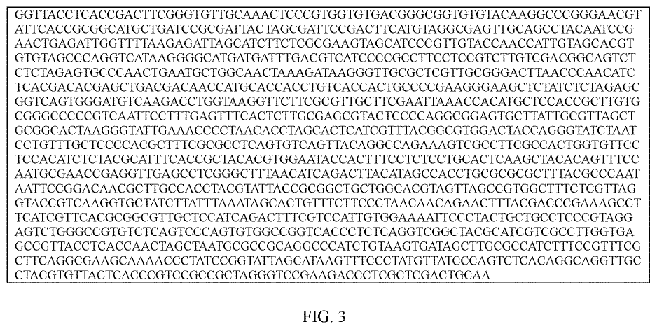

The colony morphology of strain SCUT-BR-2 is shown in FIG. 1. Its colonies on beef extract peptone medium appear milky white, approximately circular, with smooth and neat edges, a raised center, and semitransparent. Select the colonies on the plate for Gram staining, as shown in FIG. 2. Some of them are Gram negative, while others are Gram positive, with the majority being Gram negative. This is because the Gram staining reaction of Brevibacillus laterosporus is unstable: in the logarithmic phase, Gram staining is positive, and in the late stable and declining phases, Gram staining is negative.

Physiological and Biochemical Identification of Brevibacillus laterosporus

Partial physiological and biochemical characteristics of strain SCUT-BR-2 are shown in Table 1. Combined with morphological features, based on the “Handbook of Common Bacterial System Identification” and “Handbook of Berger's Bacterial System Identification”, the strain can be preliminarily identified as Brevibacillus.

TABLE 1
Physiological and biochemical test results of strain SCUT-BR-2

| Physiological and | | Physiological and | |
|---|---|---|---|
| biochemical tests | Results | biochemical tests | Results |
| Contact enzyme | + | Methyl Red | + |
| Oxidase | − | H₂S utilization | − |
| Anaerobic growth | + | Indole production | − |
| Nitrate reduction | + | 15° C. grow | − |
| Gelatin liquefaction | + | 30° C. grow | − |
| VP | − | Glucose produces acid | + |
| Starch hydrolysis | − | Glucose produces gas | − |
| Casein hydrolysis | + | Arabinose produces acid | − |
| Citric acid utilization | − | Mannitol produces acid | + |
| 2% NaCl utilization | + | Xylose produces acid | − |
| 5% NaCl utilization | − | | |

Note:
+ indicates a positive reaction, − indicates a negative reaction.

16S rDNA Sequence Analysis of Brevibacillus laterosporus

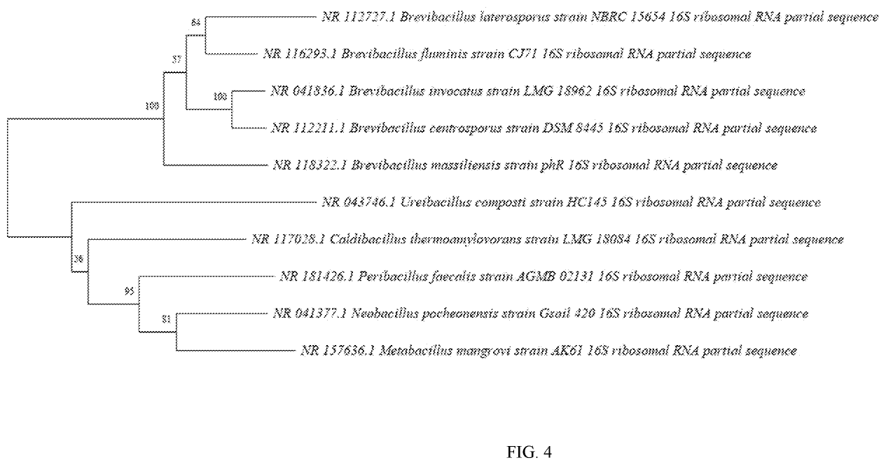

Using the bacterial genome as a template, after PCR amplification, the PCR product of the isolated strain SCUT-BR-2 showed fluorescent bands by agarose gel electrophoresis. Further PCR amplification product sequencing results showed that the 16S rDNA fragment sequence (SEQ ID NO. 1) is shown in FIG. 3. Based on this sequence, using MEGA 11.0 software, a phylogenetic tree was constructed as shown in FIG. 4. The 16S rDNA sequence analysis results showed the highest homology with a known strain Brevibacillus laterosporus (GenBank accession number NR 112727.1), with a score of 2543, a coverage rate of 99%, and a homology of 99.71%. Based on colony morphology, individual morphology, physiological and biochemical characteristics, and 16S rDNA identification results, strain SCUT-BR-2 is identified as Brevibacillus laterosporus. The strain of Brevibacillus laterosporus SCUT-BR-2 has preservation information as described above.

The collagen enzyme crude extract produced by Brevibacillus laterosporus SCUT-BR-2 proposed in the present disclosure was used to extract collagen from animal skin. The collagen enzyme crude extract was obtained by picking a certain amount of Brevibacillus laterosporus SCUT-BR-2 colonies from a beef extract peptone medium plate, adding sterile water with a bacterial mass of 1.5 times, shaking well, and centrifuging at 4° C. and 8000 rmp for 10 minutes. The supernatant obtained is the collagen enzyme crude extract used in the present disclosure.

Specific examples of extraction are as follows:

Example 1: the specific extraction method for collagen protein from fish skin using Brevibacillus laterosporus SCUT-BR-2 is as follows:

### (1) Pre-Processing

Weighing the dried fish skin, cutting it into small pieces of 3×3 cm, adding a 0.1 mol NaOH solution with 20 times the weight of the fish skin, soaking for 6 hours to remove protein and decolorize. Washing the fish skin after soaking in alkali, adding 20% ethanol solution (20 times the dry weight of the fish skin) to soak the fish skin to remove impurities such as pigments and fats, soaking for 6 hours, and then washing thoroughly. Then add 15 times the weight of the fish skin in pure water, boiling for 10 minutes, letting it cool, adjusting the pH to neutral (7.0), and setting aside for later use.

### (2) Enzyme Extraction Reaction

Adding the crude extract of collagen enzyme produced by Brevibacillus laterosporus SCUT-BR-2 according to the present disclosure to the fish skin solution at a dosage of 1% of the dry weight of the fish skin, and shaking it on a shaker for 18-36 hours. Specifically, it can be 18, 20, 22, 24, 26, 28, 30, 32, 34, 36 hours, etc., but the preferred time is 24 hours. Through experimental comparison, it was found that this preferred time can best balance efficiency and reaction quality, as well as the effective amount of enzyme in the extract.

### (3) Separation

Filter the reaction solution through a double ring qualitative filter paper, then filter it through a 0.45 μm microporous membrane. Afterwards, heat the filtrate at 70° C. for 5 minutes to inactivate the enzyme, collect the liquid, and store it frozen.

### (4) Freeze Dried Storage

Freeze and dry the extracted fish skin collagen by freeze-drying, weighing, collecting, and storing.

Example 2: using Brevibacillus laterosporus SCUT-BR-2 to extract collagen from cowhide, pigskin, and donkey skin. The specific extraction scheme is as follows:

### (1) Preprocessing

Scrape fresh cowhide, pigskin, and donkey skin, remove subcutaneous fat with a knife, and cut into 1 cm×1 cm cubes for later use. Weigh the spare skin, add a 0.1 mol NaOH solution that is 1.5 times the weight of the skin, shake and soak it on a shaker to remove fat. After 6 hours, pour out the liquid, wash the skin, and soak it in a 10% (w/w) NaCl solution that is 1.5 times the weight of the skin to remove salt soluble impurities. After soaking in saltwater for 24 hours, rinse with water to remove residual sodium chloride. Then add 1.5 times the weight of purified water, boil for 10 minutes, let it cool, adjust the pH to neutral, and set aside for later use. The wet skin quality of the spare skin is based on the mass of a wet wight of spare skin, the same applies below.

### (2) Enzyme Extraction Reaction

Add the collagen enzyme crude extract produced by Brevibacillus laterosporus SCUT-BR-2 of the present disclosure at a dosage of 1% of the skin mass, add it to an animal skin solution, and shake it on a shaker for 18-36 hours. Specifically, it can be 18, 20, 22, 24, 26, 28, 30, 32, 34, 36 hours, etc., but 24 hours is preferred. Through experimental comparison, it was found that this preferred method can best balance efficiency and reaction quality, as well as the effective amount of enzyme in the extract.

### (3) Separation

Mixing the reaction solution with 0.5-3% activated carbon powder of the obtained wet skin mass. After 3-5 hours, centrifuge at 5500 r/min for 5 minutes. Then take the supernatant and filter it through a rapid qualitative filter paper, followed by a 0.45 μm microporous filter membrane. Heat the filtrate at 70° C. for 5 minutes to inactivate the enzyme, collect the liquid, and freeze it for storage. The 0.5-3% activated carbon powder here can be preferably 1%-2% to ensure more thorough and cleaner adsorption of color and odor. However, considering the difficulty of removing activated carbon after adsorption, the amount of activated carbon powder added is more preferably 1%.

### (4) Freeze Dried Storage

Freeze and dry the collagen extracted from cowhide, pigskin, and donkey skin using freeze-drying method, weigh, collect, and store.

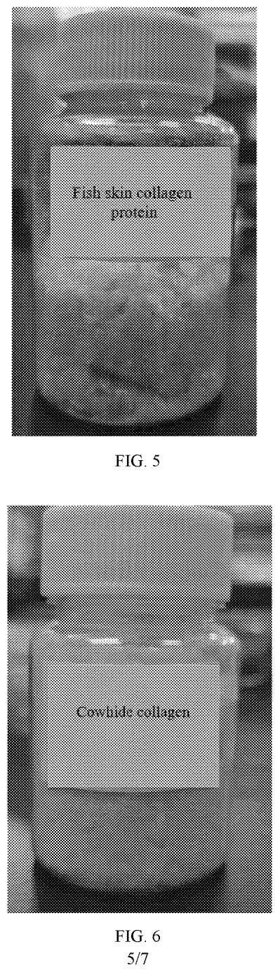

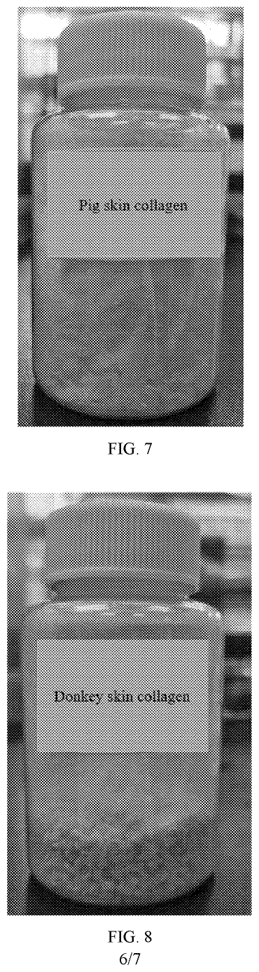

The rough appearance of the extracted samples above is shown in FIGS. 5, 6, 7, and 8.

Performance or physicochemical parameter detection of collagen extracted from various raw materials in Examples 1 and 2

### Relative Molecular Weight Detection

Mixing the extracted collagen samples with water separately to prepare a test solution with a concentration of 1 mg/mL. Shaking with a vortex shaker and let it stand for 24 hours to dissolve. Filter with a disposable sterile syringe through a 0.22 μm aqueous needle filter for later use. The chromatographic conditions for detection are the detector temperature is 25° C., the chromatographic column is Agilent 1260 Infinity II gel chromatographic column, the flow rate is 1 mL/min, and the injection volume is 10 μL.

### Infrared Spectroscopy Detection

Grind the extracted collagen sample coarse powder with potassium bromide in a mass ratio of 1:100 and press it into tablets. Use Fourier transform infrared spectroscopy (FTIR) to determine a secondary structure of the sample, and use the Shimadzu IRTracer100 instrument from Japan to obtain spectra in a range of 4000-400 cm⁻¹. Automatic signal performs 32 frequency spectral scanning at a resolution of 2 cm-1.

### Circular Column Chromatography Detection

Dissolve freeze-dried collagen in 0.1 mol/L acetic acid to a mass concentration of 0.5 mg/mL, and record a Circular Dichroism, (CD) curve of the extract in the range of 190-240 nm using a colorimetric dish with a path length of 0.1 cm-1.

### Test Results

### (1) Yield of Collagen Protein

The yield of collagen is calculated according to formula 1.

$$Yield ( \% ) = Product mass after freeze - drying / Leather mass * 100 \% \qquad ( formula 1 )$$

The collagen yields of four types of animal skin can be obtained, as shown in Table 2.

TABLE 2
Collagen yield of four animal skins

| | Dried fish | | Pork | Donkey |
|---|---|---|---|---|
| | skin | Cowhide | skin | skin |
| Leather mass (g) | 10.40 | 173.23 | 85.36 | 83.2 |
| Product mass after freeze- | 4.12 | 21.96 | 7.26 | 12.43 |
| drying (g) | | | | |
| Yield (%) | 39.63 | 12.68 | 8.51 | 14.94 |

### 2) Relative Molecular Weight of Collagen Protein

The Relative Molecular Weights of Various Types of Collagens are Shown in Table 3.

TABLE 3
Collagen GPC results of four animal skins

| | Dried | Cow | Pork | Donkey |
|---|---|---|---|---|
| | fish skin | hide | skin | skin |
| Number-average molecular | 67 | 31 | 123 | 26 |
| weight (g/mol) | | | | |
| Weight-average molecular | 4580 | 3035 | 4633 | 3454 |
| weight (g/mol) | | | | |
| Dispersion coefficient | 68.36 | 97.90 | 37.67 | 132.85 |

### (3) Infrared Spectroscopy Detection Results of Collagen Protein

The infrared spectrum of collagen can obtain amide A band, B band, I band, II band, and III band.

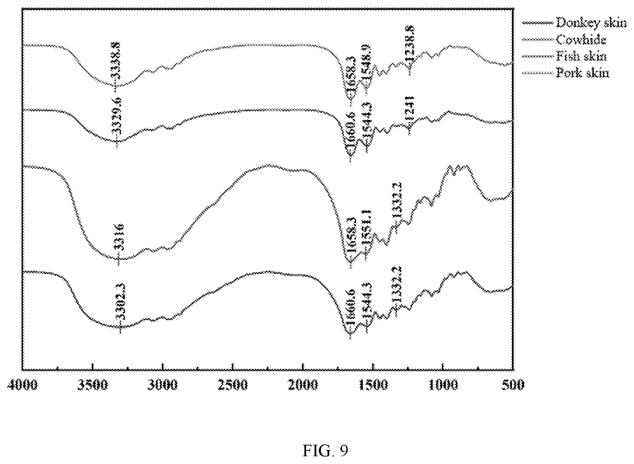

FIG. 9 shows the infrared spectra of four extracted collagen proteins detected by FTIR. All four samples exhibit characteristic absorption peaks of collagen, including amide A band, amide I band, amide II band, and amide III band. The amide A band is the strongest absorption band in amide bonds, which is related to a stretching vibration of the N—H bond. A free stretching vibration of the N—H bond occurs in a range of 3300-3500 cm⁻¹. The amide A bands of collagen from dried fish skin, cowhide, pig skin, and donkey skin are located at 3329.6 cm⁻¹, 3316 cm⁻¹, 3338.8 cm⁻¹, and 3302.3 cm⁻¹, respectively. The amide B band is located in a range of 1680-1780 cm⁻¹, mainly generated by C—O stretching vibration and N—H bending vibration, representing an asymmetric extension of CH₂ functional group, which is a characteristic functional group of a tertiary structure. The amide I band originates from C—O bond stretching vibration, N—H bond bending vibration, C—N bond stretching, and C—CN bond deformation, and is commonly used to reflect the secondary structure of proteins. Previous studies have shown that the positions of the following secondary structural regions in amide I are: β-turning, 1640-1660 cm⁻¹; Alpha helix, 1650-1660 cm⁻¹; irregular curling, 1640-1650 cm⁻¹; and β-folding, 1600~1640 cm⁻¹. The amide I bands of collagen from dried fish skin, cowhide, pig skin, and donkey skin are located at 1660.6 cm⁻¹, 1658.3 cm⁻¹, 1658.3 cm⁻¹, and 1660.6 cm⁻¹, respectively. The stretching vibration of amide II band (1510-1580 cm⁻¹) corresponds to the stretching vibration of C—H and N—H bonds. The amide II bands of collagen from dried fish skin, cowhide, pig skin, and donkey skin are located at 1544.3 cm⁻¹, 1551.1 cm⁻¹, 1548.9 cm⁻¹, and 1544.3 cm⁻¹, respectively. The amide III band is located at 1200-1335 cm⁻¹, reflecting the absorption caused by the vibration peak of the CH₂ group of the glycine main chain and proline side chain, which is related to the integrity of the triple helix structure of collagen.

### (4) Circular Dichroism Detection Results of Collagen Protein

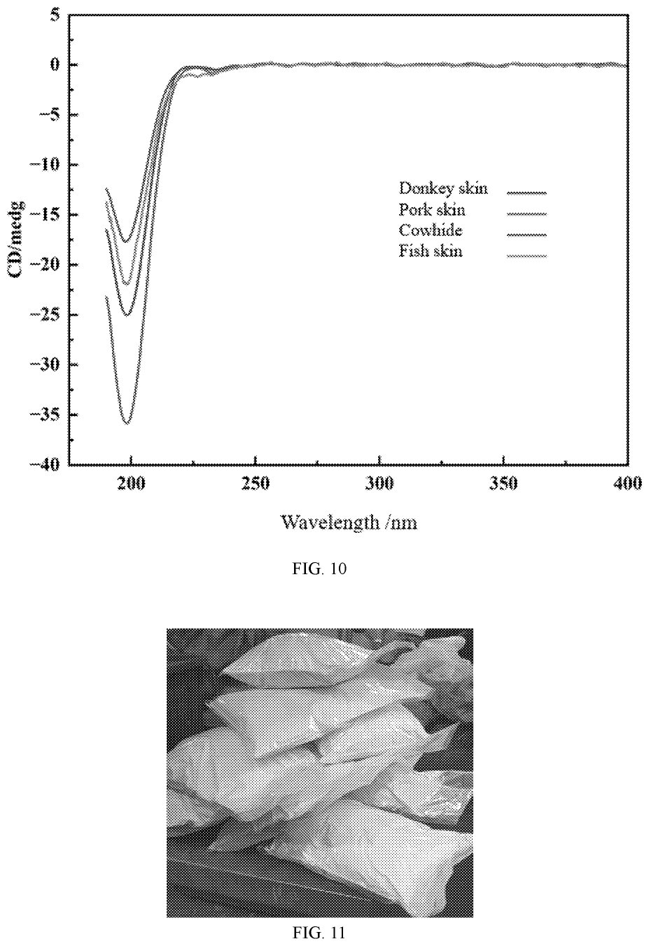

FIG. 10 shows the CD (Circular dichroism) spectra of collagen extracted from four animal skins. It can be seen from FIG. 10 that collagen has characteristic absorption peaks at 198 and 225 nm. The negative absorption peak at a wavelength of 198 nm is a typical feature of the irregular curling structure in the conformation of collagen molecules, while the positive absorption peak at 225 nm is a typical feature of the left-handed poly (proline) (P-II) peptide chain configuration. The characteristic peak of collagen at 225 nm can reflect the level of collagen triple helix. These four types of collagens have a small positive absorption peak at 225 nm, indicating that there are fewer advanced triple helix structures present in collagen.

The secondary structure analysis results of collagen are shown in Table 4. Irregular curling accounts for the vast majority of the four conformations, while alpha helices have the smallest proportion, indicating that the collagen enzyme crude extracts also hydrolyzed collagen while extracting it.

TABLE 4
Secondary structure analysis results of collagen protein

| | α- | β- | β- | Irregular | |
|---|---|---|---|---|---|
| Sample Name | spiral | fold | turning | Curling | Total |
| Donkey skin collagen | 6.5% | 18.1% | 14.9% | 60.4% | 99.9% |
| protein | | | | | |
| Pig skin collagen protein | 7.3% | 11.0% | 13.2% | 68.4% | 99.9% |
| Cowhide collagen protein | 6.0% | 23.1% | 15.9% | 55.0% | 100.0% |
| Fish skin collagen | 6.5% | 20.1% | 15.5% | 57.9% | 100.0% |
| protein | | | | | |

Therefore, the crude extract of collagen protease produced by Brevibacillus laterosporus SCUT-BR-2 was screened and isolated in this application. The collagen protein was extracted from donkey skin, pig skin, cowhide, and fish skin and detected by infrared spectroscopy. It was found that the extracts all had amide specific absorption peaks. The circular dichroism spectrum showed fewer advanced triple helix structures but more irregular curing structures (a typical feature of collagen molecular conformation), which proves that the extract is collagen protein. It also proves that it is feasible to directly use the crude extract of collagen protease produced by Brevibacillus laterosporus SCUT-BR-2 to extract collagen protein from animal skin in this application. It is a substitute for expensive and complex commercial enzyme products currently available. A feasible plan for low-cost extraction of collagen.

Example 3: The technical scheme for obtaining collagen peptides by decomposing bovine bone collagen protein using Brevibacillus laterosporus SCUT-BR-2 is as follows:

### (1) Preparation of Bovine Bone Collagen Peptide

Prepare a 12.5% bovine bone collagen solution (stirred and dissolved at 50° C.), add 0.3% (based on a mass of bovine bone collagen) of collagen enzyme crude extract, and react at 50° C. for 8-24 hours, which can be 8, 10, 12, 14, 16, 18, 20, 22, 24 hours, etc., but it is more preferred to react for 12 hours because at this preferred time, the degradation rate and comprehensive experimental efficiency of bovine bone collagen are optimal. Then add 0.5%-3% activated carbon powder (based on the mass of bovine bone collagen protein) to the reaction solution, adsorb and deodorize for 1 hour, filter once with Double Circle brand qualitative rapid filter paper, and filter once with 0.45 μm microporous filter membrane to obtain a clear and transparent filtrate. Heat the filtrate at 70° C. for 5 min to inactivate the enzyme, collect the liquid and dry it with spray, and the dry solid obtained is as shown in FIG. 11. The 0.5-3% activated carbon powder here can be preferably 1%-2% to ensure more thorough and cleaner adsorption of color and odor. However, considering the difficulty of removing activated carbon after adsorption, the amount of activated carbon powder added is more preferably 1%.

The specific sample volume of spray drying is 40-110 L/h, an inlet air temperature is 120-240° C., preferably 60-90 L/h, and the inlet air temperature is 150-200° C. For example, the sample volume can be 60, 65, 70, 75, 80, 85, 90 L/h, and the inlet air temperature can be 150, 160, 170, 180, 190, 200° C., etc. Under the most preferred sample amount of 70 L/h and air inlet temperature of 180° C., the spray drying effect is the best, which is shown in that the spray drying efficiency is high, the amount of particles adhering to an inner wall of equipment is the least, the yield is high, the process control process is optimal, and the uniformity of the product particles of bovine bone collagen peptide with certain properties and molecular weight obtained under the enzymatic reaction of the application is better.

### Relative Molecular Weight Detection of Bovine Bone Collagen Peptides

Mix the bovine bone collagen peptide samples separately with the mobile phase solution to prepare a test solution with a concentration of 1 mg/mL. Shake with a vortex shaker and let it stand for 24 hours to dissolve. Filter with a disposable sterile syringe through a 0.22 μm aqueous needle filter for later use.

Chromatographic conditions are as follows: the detector temperature is 25° C., the chromatographic column is Agilent 1260 Infinity II gel chromatographic column, the flow rate is 1 mL/min, and the injection volume is 40 μL.

### Activity Detection of Bovine Bone Collagen Peptide

DPPH radical scavenging rate

1,1-diphenyl-2-trinitrophenylhydrazine (DPPH) is a stable long-life free radical, with a deep purple color in ethanol solution and strong absorption around 517 nm. When there is a free radical scavenger present, the light absorption of DPPH ethanol solution is weakened due to pairing with a single electron. The degree of fading of DPPH ethanol solution is linearly related to the number of electrons it accepts, which can be used to evaluate the ability of the test sample to eliminate free radicals.

Weigh a quantitative sample, dilute it with water to obtain a multi-level concentration sample, shake/beat thoroughly during the reaction, mix well, and then let it stand. Dissolve DPPH reagent in 95% ethanol to prepare 0.12 mg/mL, and prepare DPPH working solution in the dark. Trolox uses 95 ethanol to prepare mother liquor with a concentration of 0.08 mg/mL, which is diluted 1.5 times to form a series of concentration gradients for validating the experimental system. The concentration settings should include 0.01, 0.02, 0.04, and 0.08 mg/mL as positive controls. Referring to the sample liquid addition grouping list, a 96 well plate was used for the experiment, and a sample group (T), a sample background group (TO), a negative control group (C), and a solvent background group (CO) were established. The positive control group was treated the same as the sample group. Set up 3 parallel teas for each group. Add reagents in sequence using a pipette to ensure consistent reaction time.

TABLE 5
Grouping list of antioxidant test samples with liquid addition

| | T- | T0-Sample | C-blank | C0-Solvent |
|---|---|---|---|---|
| | sample | background | control | background |
| | group | group | group | group |
| Sample solution (μL) | 50 | 50 | — | — |
| Water (μL) | 100 | 100 | 150 | 150 |
| DPPH ethanol solution | 50 | — | 50 | — |
| (μL) | | | | |
| 95 ethanol (μL) | — | 50 | — | 50 |

After 5 minutes of light avoidance reaction, immediately use an enzyme-linked immunosorbent assay (ELISA) reader to detect. The parameters are set to slow shake for 10 seconds and measure the absorbance at OD517 nm. Export the file after the instrument completes the reading.

The formula for calculating the DPPH radical scavenging rate is as follows:

$$Scavenging rate = ( 1 - \frac{T - {T}_{0}}{C - {C}_{0}} ) \times 100 \% \qquad ( formula 2 )$$

### Elastase Inhibition Rate

Elastin plays a crucial role in the youthfulness of the skin, and its deficiency is the main cause of skin aging. Inhibiting the activity of elastase can effectively slow down the breakdown of elastin and maintain the elasticity of the skin. Elastase can catalyze the hydrolysis of hydroxyl groups in peptide bonds of various amino acids. Therefore, when reacting with the substrate N-succinyl-alanine-alanine-p-nitroaniline, colored substances are produced, and their absorbance values can be measured at 410 nm using a spectrophotometer. Based on the changes in absorbance values, the inhibitory effect of the test substance on elastase activity can be calculated to evaluate whether the test substance has firming and anti-wrinkle effects.

Referring to the sample addition grouping list, a 96 well plate was used for the experiment, with sample group (T), sample background group (TO), enzyme reaction group (C), and solvent background group (CO) established. The positive control group was treated the same as the sample group. Set up 3 parallel tests for each group.

TABLE 6
Grouping list of firming and anti-wrinkle
test samples with liquid addition

| | | T0-Sample | C-blank | C0-Solvent |
|---|---|---|---|---|
| | T-sample | background | control | background |
| | group | group | group | group |
| Elastase | + | − | + | − |
| Positive control, | + | + | − | − |
| test substance | | | | |
| Buffer solution | − | + | + | + |
| | | 25° C., 15 min | | |
| N-Succinyl- | + | + | + | + |
| Ala-Ala-Ala-p- | | 25° C., 15 min | | |
| nitroanilide, | | | | |
| (AAAPAN) | | | | |

Immediately use an enzyme-linked immunosorbent assay (ELISA) reader for detection, with parameters set to slow oscillation for 10 seconds and OD410 nm absorbance measurement. Export the file after the instrument completes the reading.

The method for calculating the inhibition rate of elastase activity is as follows:

$$Inhibition rate = ( 1 - \frac{T - {T}_{0}}{C - {C}_{0}} ) \times 100 \% \qquad ( formula 3 )$$

### Hyaluronic Acid Inhibition

Hyaluronic acid, as an important natural moisturizing substance, plays a crucial role in maintaining the volume of extracellular matrix and normal physiological functions of the skin. However, hyaluronic acid is specifically broken down by hyaluronidase, and its products promote the inflammatory response of the skin, leading to an increase in histamine production in the body. Besides that, hyaluronidase expression is also associated with most IgE mediated type I and T cell-mediated type IV allergic reactions. Inhibiting the activity of hyaluronidase is beneficial to some extent in suppressing the development of skin inflammation and allergic reactions, and improving the stimulating state of the skin caused by inflammation and allergic reactions. Therefore, inhibiting hyaluronidase activity is often used as an important indicator for evaluating the soothing and moisturizing properties of products. Hyaluronidase breaks the glycosidic bond between N-acetylglucosamine and D-glucuronic acid in the HA sugar chain of hyaluronic acid, generating a reducing sugar that can reduce DNS to an amino compound under alkaline conditions. After boiling in water, it can be fully colored, and its absorbance can be measured at 540 nm to calculate hyaluronidase activity.

Reagents and materials: hyaluronidase, hyaluronic acid, positive control: dexamethasone, DNS reagent.

Refer to the sample liquid addition grouping list and conduct the experiment using a 2 mL centrifuge tube. Each group has 3 parallel tubes.

TABLE 7
Group list of soothing and moisturizing
test samples with liquid addition

| | | T0-Sample | C-blank | C0-Solvent |
|---|---|---|---|---|
| | T-sample | background | control | background |
| | group | group | group | group |
| Hyaluronidase | + | − | + | − |
| Hyaluronic acid | + | + | + | + |
| Positive control, | + | + | − | − |
| test substance | | | | |
| Buffer solution | − | + | + | + |
| | | 37° C., 30 min | | |
| DNS | + | + | + | + |
| | | Boiling water | | |
| | | bath, 5 min | | |

After the test tube cools down, shake well and add 150 μL of reaction solution to a 96 well plate. Use an enzyme-linked immunosorbent assay (ELISA) reader to measure the absorbance at OD540 nm.

The method for calculating the inhibition rate of hyaluronidase activity is as follows:

$$Inhibition rate = ( 1 - \frac{T - {T}_{0}}{C - {C}_{0}} ) \times 100 \% \qquad ( formula 3 )$$

### Yield of Bovine Bone Collagen Peptide

The bovine bone collagen peptide obtained after spray drying is shown in FIG. 11. According to the yield formula, the yield of bovine bone collagen peptide is

$$Yield = 8.496 kg / 20 kg * 100 \% = 42.48 \%$$

### Relative Molecular Weight Detection Results of Bovine Bone Collagen Peptide

The weight average molecular weight of the bovine bone collagen peptide is 1870 g/mol, and the dispersion coefficient is relatively close to 1, indicating that the molecular weight distribution of bovine bone collagen peptide is relatively uniform after the collagen enzyme of Brevibacillus laterosporus SCUT-BR-2 decomposes bovine bone collagen.

TABLE 8
GPC detection results of the bovine bone collagen peptide

| | Bovine bone collagen peptide |
|---|---|
| Number-average molecular weight | 1237 |
| Weight-average molecular weight | 1870 |
| Dispersion coefficient | 4.51 |

### Activity Detection Results of Bovine Bone Collagen Peptide

The activity detection results of collagen peptides are shown in Table 9. 1% (W/V) of the test substance collagen peptide has a scavenging effect on DPPH free radicals, with a scavenging rate of 10.53±0.47%, and the data shows significant differences (P=0.0003). This indicates that the experimental results can serve as evidence to support the evaluation of the antioxidant effect of collagen peptides by scavenging DPPH free radicals. 2% (W/V) of the test substance collagen peptide has an inhibitory effect on elastase catalyzed degradation of AAAPAN, with an inhibition rate of 14.39±0.52%, and the data shows significant differences (p=0.0280). This indicates that the experimental results can serve as evidence to support the effect of collagen peptides on tightening and wrinkle resistance by inhibiting elastase activity. 2% (V/V) of the test substance collagen peptide has an inhibitory effect on the degradation of hyaluronic acid by hyaluronidase, with an inhibition rate of 47.02±2.49%, and the data shows significant differences (p=0.0025). This indicates that the experimental results can serve as evidence to support the soothing and moisturizing effects of the test substance collagen peptide by inhibiting hyaluronidase activity.

TABLE 9
Activity detection results of bovine bone collagen peptides

| | Blank | Positive | |
|---|---|---|---|
| | group | control | Sample |
| PPH radical | 0.00 ± 1.48 | 55.47 ± 0.23**** | 10.53 ± 0.47*** |
| scavenging rate (%) | | | |
| Inhibition rate of | 0.00 ± 3.75 | 70.70 ± 0.88** | 14.39 ± 0.52* |
| elastase activity | | | |
| Inhibition rate of | 0.00 ± 1.70 | 51.66 ± 1.52*** | 47.02 ± 2.49** |
| hyaluronic acid | | | |
| activity | | | |

Compared to blank group:
*p < 0.05;
**p < 0.01;
***p < 0.0001.

Where the positive control for DPPH radical scavenging rate is 40 μg/mL vitamin E, and the sample is 1% (W/V) bovine bone collagen peptide; the positive control for elastase activity inhibition rate was 200 μg/mL EGCG, and the sample was 2% (W/V) bovine bone collagen peptide. The positive control for the inhibition rate of hyaluronidase activity was 0.5 mg/mL dexamethasone sodium phosphate, and the sample was 2% (V/V) bovine bone collagen peptide.

The above tests indicate that the Bacillus subtilis SCUT-BR-2 provided by the present disclosure has a significant effect on decomposing bovine bone collagen into small molecule peptides, and the obtained bovine bone collagen peptide has a DPPH free radical scavenging rate of 10.53±0.47%, an elastase inhibition rate of 14.39±0.52%, and a hyaluronidase inhibition rate of 47.02±2.49%, indicating that bovine bone collagen peptide has certain antioxidant, anti-wrinkle and firming effects, and soothing and moisturizing effects. Therefore, it is completely feasible to use the bovine bone collagen peptide in skin care products, such as cream, lotion, essence, skin care lotion, facial mask, etc., which can fight free radicals, replenish water, soothe and moisturize.

The above embodiments only express partial embodiments of the present disclosure, and their descriptions are more specific and detailed, but should not be understood as limiting the scope of the present disclosure. It should be noted that for those skilled in the art, several modifications and improvements can be made without departing from the inventive concept, which are within the protection scope of the present disclosure. Therefore, the protection scope of the present invention should be based on the appended claims.

What is claimed is:

1. A Brevibacillus laterosporus, named as Brevibacillus laterosporus SCUT-BR-2, was deposited on Oct. 15, 2024, it was deposited at the Guangdong institute of microbiology, with an address at 5th Floor, No. 59 Building, No. 100 Xianlie Zhong Road, Yuexiu District, Guangzhou 510070, PR China and with a deposit number of GDMCC No. 65271;
the Brevibacillus laterosporus strain is isolating, culturing, and purifying from meat products;
a culture medium used for Brevibacillus laterosporus includes a LB medium, a medium A, and a beef extract peptone medium;
a formula of the culture medium A is: collagen powder 0.8%, beef paste 0.1%, glucose 0.1%, KH₂PO₄ 0.05%, MgSO₄·7H₂O 0.01%, Agar 2%, adjusting a pH of medium A to 7.0;
wherein steps of isolating, culturing, and purifying Brevibacillus laterosporus from meat products include: grinding the meat products, weighing 1 g and adding it to a triangular flask containing 49 mL sterile physiological saline, shaking thoroughly for 30 minutes, standing still, taking a middle supernatant as a bacterial suspension, and performing gradient dilution, gradient diluted bacterial suspension was separately coated onto a LB agar solid medium using plate dilution separation method, and cultured in a constant temperature incubator at 30-40° C. until single colonies grew, selecting single colonies and placing on a screening medium A plate, and inverting and cultivating for 1-2 days at 30-40° C., based on screening characteristics, selecting single colonies with normal growth and significant differences in colony morphology and repeatedly streaking on beef extract peptone medium for pure culture, after numbering, they were stored for future use.

2. A method for enzymatic hydrolysis of bovine bone collagen using Brevibacillus laterosporus according to claim 1, comprises: preparing a 12.5% bovine bone collagen solution, stirring and dissolving at 50° C., adding 0.3% based on a mass of bovine bone collagen of collagen enzyme crude extract, reacting at 50° C. for 8-24 hours, then adding 0.5%-3% activated carbon powder based on the mass of bovine bone collagen to a reaction solution, adsorbing and deodorizing for 1 hour, filtering once with Double Circle brand qualitative rapid filter paper, and then filtering once with a 0.45 μm microporous filter membrane to obtain a clear and transparent filtrate; heating the filtrate at 70° C. for 5 min to inactivate an enzyme, collecting the filtrate for spray drying to obtain the bovine bone collagen peptide.

3. The method according to claim 2, wherein the bovine bone collagen peptide has at least one of the following properties:
(1) a scavenging rate of bovine bone collagen peptide on DPPH free radicals is 10.53±0.47%;
(2) an inhibition rate of elastase is 14.39±0.52%;
(3) an inhibition rate of hyaluronidase is 47.02±2.49%;
(4) a weight average molecular weight of bovine bone collagen peptide is 1870 g/mol.

4. The method according to claim 3, wherein the collagen enzyme crude extract is obtained by the following method: picking a certain amount of Brevibacillus laterosporus SCUT-BR-2 colonies from a beef extract peptone medium plate, adding sterile water with a bacterial mass of 1.5 times, shaking well, and centrifuging at 4° C. and 8000 rmp for 10 minutes, an obtained supernatant is the collagen enzyme crude extract used.

5. The method according to claim 4, wherein a yield of bovine bone collagen peptide obtained is 42.48%.

6. A use of bovine bone collagen peptide prepared by claim 2 in skincare products.

7. A use of a crude collagen enzyme extract produced by Brevibacillus laterosporus according to claim 1 in an extraction of collagen from fish skin, pig skin, cowhide, and donkey skin.


SEQUENCE LISTING (1 sequence)

SEQ ID NO: 1 - DNA, 1388 bp, synthetic construct
  source 1..1388: mol_type = other DNA
ggttacctcaccgacttcgggtgttgcaaactcccgtggtgtgacgggcggtgtgtacaaggcccgggaacgtattcaccgcggcatgctgatccgcgattactagcgattccgacttcatgtaggcgagttgcagcctacaatccgaactgagattggttttaagagattagcatcttctcgcgaagtagcatcccgttgtaccaaccattgtagcacgtgtgtagcccaggtcataaggggcatgatgatttgacgtcatccccgccttcctccgtcttgtcgacggcagtctctctagagtgcccaactgaatgctggcaactaaagataagggttgcgctcgttgcgggacttaacccaacatctcacgacacgagctgacgacaaccatgcaccacctgtcaccactgccccgaagggaagctctatctctagagcggtcagtgggatgtcaagacctggtaaggttcttcgcgttgcttcgaattaaaccacatgctccaccgcttgtgcgggcccccgtcaattcctttgagtttcactcttgcgagcgtactccccaggcggagtgcttattgcgttagctgcggcactaagggtattgaaacccctaacacctagcactcatcgtttacggcgtggactaccagggtatctaatcctgtttgctccccacgctttcgcgcctcagtgtcagttacaggccagaaagtcgccttcgccactggtgttcctccacatctctacgcatttcaccgctacacgtggaataccactttcctctcctgcactcaagctacacagtttccaatgcgaaccgaggttgagcctcgggctttaacatcagacttacatagccacctgcgcgcgctttacgcccaataattccggacaacgcttgccacctacgtattaccgcggctgctggcacgtagttagccgtggctttctcgttaggtaccgtcaaggtgctatcttatttaaatagcactgtttcttccctaacaacagaactttacgacccgaaagccttcatcgttcacgcggcgttgctccatcagactttcgtccattgtggaaaattccctactgctgcctcccgtaggagtctgggccgtgtctcagtcccagtgtggccggtcaccctctcaggtcggctacgcatcgtcgccttggtgagccgttacctcaccaactagctaatgcgccgcaggcccatctgtaagtgatagcttgcgccatctttccgtttcgcttcaggcgaagcaaaaccctatccggtattagcataagtttccctatgttatcccagtctcacaggcaggttgcctacgtgttactcacccgtccgccgctagggtccgaagaccctcgctcgactgcaa